(19) United States
(12) Patent Application Publication
(10) Pub. No.: US 2005/0014823 A1
(43) Pub. Date: Jan. 20, 2005

(54) Topical anesthetic composition and method of administration

(76) Inventors: Patrick L. Soderlund, New Prague, MN (US); Raymond E. Faulk, Mullins, SC (US)

Correspondence Address: Paul W. Busse;FAEGRE & BENSON LLP, 2200 Wells Fargo Center, 90 South Seventh Street, Minneapolis, MN, 55402-3901, US

(21) Appl. No.: 10/621,703

(22) Filed: Jul. 17, 2003

(51) Int. Cl. A61K 31/46; A61K 31/24

(52) U.S. Cl. 514/536; 514/304

(57) ABSTRACT

The present invention includes a topical anesthetic composition having one or more local anesthetic agents, a penetration enhancer, and an anhydrous, volatile solvent combined to provide a liquid homogenous solution. The present invention may also include water in the topical anesthetic composition as concentration levels low enough to avoid precipitation of the local anesthetic agent and penetration enhancer from solution and undesired enhanced delivery of the local anesthetic agent through the skin or mucosa to the systemic circulation. The topical anesthetic composition can be conveniently sprayed onto intact skin and can be absorbed percutaneously with greater efficacy in order to shorten the time needed for the anesthesia to take effect.

### BACKGROUND OF THE INVENTION

Fear of needles, belonephobia, is a very real and common condition. And even absent the more extreme condition of belonephobia, few people look forward to obtaining a shot. With modern medicine becoming more and more reliant upon the use of needles for blood tests and the administration of drugs, however, the fear of needles is becoming an increasingly important issue for health care professionals and their patients. Extreme or irrational fear of needles can keep people from receiving the medical treatment they need, which may result in serious, sometimes irreversible, damage. Topical administration of a local anesthetic agent, however, can be accomplished without needles to anesthetize the intended area for subsequent procedures. Accordingly, there is a need for a safe and effective local anesthetic composition that can be applied topically to ease pain during dermal procedures, such as venipuncture, intravenous cannulation, punch biopsy and other small incisions, vaccination, and circumcision.

Anesthesia is a partial or complete loss of sensation or feeling induced by the administration of various substances. There are many different types of anesthesia but they are usually placed into one of three groups. These groups are general anesthesia, local anesthesia, and spinal anesthesia. General anesthetics act primarily on the brain, rendering people both insensible to pain and unconscious. Local anesthetics affect only part of the body and the patient remains conscious. Local anesthetics are usually administered through a gel or cream on the surface of the skin or mucosa but can also be injected underneath the skin. When local anesthetics are applied directly to the skin or mucosa they are also referred to as topical anesthetics. Topical anesthetics are absorbed through the skin or mucosa so that they can interact with nerve endings within the dermis. Once absorbed, topical anesthetics cause a depolarization of sensory nerves within the outer dernis, which temporarily deactivates these nerves. While the anesthetic effect is present, the deactivated sensory nerves do not transmit impulses to the brain. Painful sensations within the anesthetized area are thus temporarily decreased or eliminated.

A number of topical anesthetic compositions with acceptably low concentrations of a local anesthetic agent are known to produce satisfactory results when applied specifically to mucosa. These topical anesthetic compositions typically contain local anesthetic agents such as lidocaine, which are readily absorbed from the gastrointestinal tract, from mucosa, and through damaged skin. When topically applied directly to the mucosa, these topical anesthetic compositions act rapidly and the anesthetic effects last over the duration of intended use. Absorption of the local anesthetic agent through intact skin, however, is generally poor and thus the efficacy of these topical anesthetic compositions in intact skin applications has been much less satisfactory.

Eutectic compounds have been designed to enhance membrane transport of drugs through improved solubility and absorption. A eutectic mixture of local anesthetics (EMLA), when applied topically, penetrates intact skin after an application period of one to two hours. For example, EMLA cream (Astra Pharmaceuticals, Inc., Westboro, Mass.), (U.S. Pat. No. 4,562,060 and U.S. Pat. No. 4,529,601) is a topical anesthetic product currently marketed in many countries, including the United States. Because EMLA cream contains a relatively high concentration of local anesthetic in its oil phase, it exhibits improved efficacy on intact skin compared with other conventional topical anesthetic formulations, which are effective only on mucosa.

A disadvantage of EMLA cream, however, includes the mess and inconvenience of having to apply the cream one to two hours before the anesthetic effects are realized. There also exists variability in the in the amount of anesthetic agent imparted to the intended area. Further, EMLA cream contains prilocalne, which presents the risk of methemoglobinemia, a serious condition characterized by the ferric form of hemoglobin with impaired oxygen-carrying capacity that results upon metabolization of prilocalne (B. Jakobson et al., Acta Anaesthesialogica Scandinavica 29: 453-455 (1985)). Therefore, the use of EMLA in young children has been severely restricted.

The anesthetic agent lidocaine is a widely used local anesthetic agent. Due to low permeability of lidocaine through the stratum corneum, however, the efficacy of lidocaine alone for topical application of local anesthetic agents through intact skin has been extremely disappointing to date. Conventional lidocaine creams may be readily prepared by simply dissolving lidocaine in a suitable pharmaceutical oil and emulsifying the components, but these creams can not effectively deliver lidocaine for transdermal anesthesia on intact skin. For example, in medical facilities such as hospitals, a topical anesthetic is sometimes prepared and used in accordance with a method in which an active anesthetic ingredient is blended with, for example, an agent of ointment, cream, or gel to prepare a clinically formulated topical anesthetic. The topical anesthetic is tightly sealed upon administration with an extremely air-tight resin film such as polyvinylidene chloride film. This method is referred to as the “ODT Method”; (Hifu, 34 (2), 237-242 (1992)). It takes about two hours to obtain a sufficient anesthetic effect after application of the cream using the ODT method. Efficacy can only be achieved when the concentration of lidocaine in the cream or gel for topical application is unacceptably high (e.g., greater than about 30% by weight). Such high concentrations of lidocaine pose a risk of systemic toxicity. Limited by the intrinsic solubility of lidocaine in pharmaceutical oils, lidocaine concentration in the oil phase of conventional creams cannot readily reach the concentration that is necessary for effective transdermal delivery.

Some of the clinically prepared local anesthetic compositions also have poor stability, and hence it is necessary to prepare the local anesthetic compositions just before they are to be used. This can make the local anesthetic compositions inconvenient to use. Further, some of the topical anesthetics are administered using a tape-shaped medicament, which is unsuitable to anesthetize a broad skin surface.

U.S. Pat. No. 6,429,228 (Inagi et al.) reports a local anesthetic gel for topical application having an improved efficacy. Effectiveness of the local anesthetic gel was reported as being realized 30 minutes after application on the skin of the animal being tested. The reported local anesthetic gel is formulated using an active ingredient selected from lidocaine, prilocalne, and pharmaceutically acceptable salts thereof, a fatty acid penetration enhancer having 8-18 carbon atoms such as caprylic acid and oleic acid, ethanol and/or isopropyl alcohol and water. The gel form of the local anesthetic, however, remains inconvenient to use and apply and the efficacy, while improved over EMLA cream, still requires the user to wait for 30 minutes.

Therefore, there exists a need for a topical anesthetic composition that provides fast, convenient and reliable delivery of anesthesia for minor interventions on intact skin, such as blood sampling and administration of medication by injection without the risk of systemic toxicity. Further, a topical anesthetic composition containing lidocaine but little or no prilocalne would have a significant clinical advantage over EMLA and would also expand the use of topical anesthetic compositions in children and particularly in infants and newborns.

### SUMMARY OF THE INVENTION

The present invention includes a topical anesthetic composition having one or more local anesthetic agents, a penetration enhancer, and an anhydrous volatile solvent combined to provide a liquid homogenous solution. The topical anesthetic composition can be sprayed onto intact skin and can be absorbed percutaneously with greater efficacy in order to shorten the time needed for the anesthesia to take effect.

In one embodiment of the present invention, the topical anesthetic composition contains a local anesthetic agent in a concentration of from about 5 to 50 wt %, a penetration enhancer in a concentration of from about 1 to 30 wt %, an anhydrous volatile solvent in a concentration of from about 10 to 94 wt % and from about 0.01 to 20 wt % water. In an alternative embodiment of the present invention, the local anesthetic agent is lidocaine, the penetration enhancer is a fatty acid ester such as isopropyl palmitate, and the anhydrous volatile solvent is ethanol.

The amount of water is limited to minimize both occlusion and the possibility of the local anesthetic agent and/or the penetration enhancer precipitating out of solution. Therefore, in an embodiment of the present invention, the concentration of water is less than about 10 wt %. In yet another embodiment of the present invention the concentration of water is less than about 5 wt %. In yet another embodiment of the present invention, the concentration of water is 0.01 wt %.

The topical anesthetic composition of the present invention is in the form of a liquid homogenous solution that can be sprayed directly onto the exposed intact skin surface, to anesthetize the intact skin surface and ease the pain during dermal procedures, such as venipuncture, intravenous cannulation, punch biopsy and other small incisions, vaccination, and circumcision. In an alternative embodiment, the topical anesthetic composition can also be sprayed onto mucosa.

### DETAILED DESCRIPTION OF THE INVENTION

A topical anesthetic composition in the form of a liquid homogeneous solution for percutaneous administration of an anesthetic having improved efficacy, convenience, and reliability and a method of anesthetizing tissue such as intact skin and mucosa are provided. In an embodiment of the present invention, the topical anesthetic composition contains a local anesthetic agent, a penetration enhancer, an anhydrous volatile solvent and water.

The term “mammal” as used herein is intended to include all warm-blooded mammals, preferably humans.

The term “mucosa” as used herein means any moist anatomical membrane or surface on a mammal such as oral, buccal, vaginal, rectal, nasal or ophthamalic surfaces.

The term “topical” or “topically” is used herein in its conventional meaning as referring to direct contact with an anatomical site or surface area on a mammal including skin, mucosa, teeth, and nails.

The term “local anesthetic agent” as used herein means an anesthetic agent that is absorbed through the skin or mucosa to interact with nerve endings within the dermis but has very little or none, to minimal, systemic effect and is not intended for systemic use.

The term “therapeutically effective” as used herein means an amount of local anesthetic agent sufficient to achieve the desired local effect or result but no or minimal systemic effect, when applied topically, over the duration of intended use.

The term “excipient” as used herein refers to an inert substance combined with an active agent such as a local anesthetic agent or penetration enhancer to prepare a convenient dosage form and vehicle for delivering the active agent.

The term “efficacy” as used herein refers to the effectiveness of the topical anesthetic composition measured by the rate at which the anesthetic effects are observed after application of the topical anesthetic composition to the intended surface.

Local Anesthetic Agent

In one embodiment of the present invention, the local anesthetic agents are provided in base form and are selected from the group of local anesthetic agents such as lidocaine (also known as lignocaine), tetracaine, benzocaine, procaine, mepivacaine, bupivacaine, etidocaine, or cocaine. The local anesthetic agent can be a single agent or a combination of agents provided the combination provides an efficacy equivalent to pure lidocaine.

Lidocaine is 2-diethylamino-N-[2,6-dimethylphenyl]acetamide and is available under the tradename XYLOCAINE. Tetracaine is 2-dimethylaminoethyl-ethyl-p-butylaminobenzoate and is available under the tradename PONTOCAINE (Abbott Laboratories, Limited, Abbott Park, Ill.). Prilocalne is 2-propylamino-N-(2-tolyl)propionamide and is available under the tradename CITANEST (Vidal). Procaine is 2-diethylaminoethyl-p-aminobenzoate and is available under the tradename of AMINOCAINE. Mepivacaine is N-(2,6-Dimethylphenyl)-1-methyl-2-piperidinecarboxamide and is available under the tradename CARBOCAINE (Vidal). Benzocaine is 4-aminobenzoic acid ethyl ester and is available under the tradename AMERICAINE. Bupivacaine is 1-Butyl-N-(2,6-dimethylphenyl)-2-piperidinecarboxamide and is available under the tradename MARCAINE (Vidal). Etidocaine is 2-ethylpropylamino-2,6-butyroxylidide and is available under the tradename DURANEST (Astra Zeneca, London, United Kingdom).

In accordance with one aspect of the present invention, the local anesthetic agent is lidocaine. Lidocaine works by blocking the conduction of signals along nerve fibers. Lidocaine is more selective for the smaller fibers that cause pain so its use can prevent transmission of pain signals but retain feeling such as coarse touch and temperature, which are transmitted by the larger fibers.

The amount of local anesthetic agent to be incorporated in the topical anesthetic composition will vary depending on the particular anesthetic agent selected, the desired therapeutic effect, and the duration of intended use. In one embodiment of the present invention, the concentration of the local anesthetic agent is from about 5 wt % to 50 wt % of the total composition to deliver an effective dosage amount of about 30.0 mg/spray of the local anesthetic agent where a spray pump is held 1 inch from the intended surface to cover an area of 1 in². In another embodiment of the present invention, the spray pump is held 2 inches from the intended surface to cover an area of 2 in² and deliver an effective amount of about 15.0 mg/spray of the local anesthetic agent. In yet another embodiment, the concentration of the local anesthetic agent is about 35 wt %.

Penetration Enhancer

Skin differs from soft and moist mucosa in that it contains a dense stratum corneum of keratinized cells, as well as the epidermal cell layer. Both act to restrain the percutaneous penetration of topically applied substances. Additionally, the skin has a superficial, cutaneous layer (the horny layer) which consists of flat, scalelike “squames” made up of the fibrous protein keratin.

Various methods such as use of penetration enhancers, prodrugs and superfluous vehicles, iontophorosis, phonophoresis and thermophoresis have been used to increase skin permeation of local anesthetic agents. In accordance with the present invention, the topical anesthetic composition has been supplemented with one or more penetration enhancers.

Suitable penetration enhancers have no irritancy or toxicity to the skin, as well as high enhancing effects. The penetration enhancers should also be physiochemically stable and not have pharmacologic effects and preferably should not have a disagreeable smell, color or taste.

An important criterion for selecting a suitable penetration enhancer is enhanced percutaneous delivery of the local anesthetic agent into the intact skin or mucosa with minimal undesired delivery of the local anesthetic agent through the intact skin or mucosa into the systemic circulation.

Penetration enhancers are described in detail in Remington's Pharmaceutical Sciences, Vol. 18, Mack Publishing Co., Easton, Pa. (1990), in particular Chapter 87, which is incorporated herein by reference in its entirety. Suitable penetration enhancers of the present invention include fatty acid esters such as isopropyl palmitate, isopropyl myristate, isopropyl laurate, diisopropyl adipate, ester derivatives of capric acid, lauric acid, leucinic acid, and neodecanoic acid including sodium laurate, sodium caprate, cetryl laurate, myristyl lactate, lauryl lactate, methyl laurate, oleic acid esters and oleic acid ester derivatives such as methyl, ethyl, propyl, isopropyl, butyl, vinyl and glycerylmonooleate, and those given in U.S. Pat. No. 5,082,866, particularly dodecyl (N,N-dimethylamino) acetate and dodecyl (N,N-dimethylamino) propionate, sodium oleate, sucrose monooleate, sorbitan esters such as sorbitan monolaurate and sorbitan monooleate, long chain alkyl esters of 2-pyrrolidone, particularly the 1-lauryl, 1-hexyl and 1-(2-ethylhexyl) esters of 2-pyrollidone, dodecyl (N,N-dimethylamino) acetate, dodecyl (N,N-dimethylamino) propionate, and combinations thereof. Additional penetration enhancers of the present invention include mentane, menthone, menthol, terpinene, terpinene, D-terpinene, dipentene, limonene, sefsol-318 (a medium chain glyceride) and combinations thereof.

Fatty acid esters enhance penetration by increasing lipid fluidity through formation of a solvation shell around polar head groups which then leads to a disruption of lipid packing, permeating into liposomal bilayers, thus increasing fluidity and promoting permeation of drug molecules and increasing diffusivity of the stratum corneum and the partition coefficient between the stratem corneum and vehicle of both drug and solvent.

The amount and rate of percutaneous absorption is dependent upon the selection of the penetration enhancer, the amount of the penetration enhancer used, and the type and condition of skin or mucosa being treated. The amount and rate of percutaneous absorption can be optimized for a particular condition. In one embodiment of the present invention, the penetration enhancer is isopropyl palmitate. In this particular embodiment, the amount of the penetration enhancer present in the total composition is from about 1 wt % to 30 wt %. In an alternative embodiment, the composition contains isopropyl palmitate in about 10 wt %.

Anhydrous, Volatile Solvent

The present invention also contains one or more anhydrous, volatile solvents as a topical excipient. The anhydrous, volatile solvent provides a convenient dosage form and vehicle for delivering the local anesthetic agent and penetration enhancer. Such anhydrous, volatile solvents are those known in the art, and are not toxic, pharmaceutically acceptable substances, preferably liquids, which do not substantially negatively affect the properties or the solubility of the local anesthetic agents at the concentrations used.

In one embodiment of the present invention, the anhydrous volatile solvent is ethanol. Ethanol, also referred to as ethyl alcohol, anhydrous alcohol or absolute alcohol, contains less than 1% water. In addition to its solvency power, ethanol can be used as a preservative. The evaporative properties of ethanol also impart a cool feel to the skin and provide quick delivery of the local anesthetic agent and penetration enhancer. In one embodiment of the present invention, the topical anesthetic composition is sprayed onto the skin or mucosa. The ethanol quickly evaporates leaving the penetration enhancer and local anesthetic agent to work on the intended tissue.

Other suitable volatile solvents include alcohols such as 2-propanol, ketones such as acetone, alkyl methyl sulfoxides such as dimethyl sulfoxide, and alkanoic acid esters such as ethyl acetate, n-propyl acetate, isobutyl acetate, n-butyl acetate isobutyl isobutyrate, hexyl acetate, 2-ethylhexyl acetate or butyl acetate, and combinations and mixtures thereof.

The amount of the solvents used in the present invention depends on the nature and amount of the other components or ingredients. In one embodiment of the present invention, the total amount of solvent used is from about 10 wt % to 94 wt %. In an alternative embodiment of the present invention, the total amount of solvent used is from about 50 to 55 wt %.

Water

Water can also be added as a topical excipient to the present invention to provide a convenient dosage form and vehicle for delivering the local anesthetic agent and penetration enhancer. Because water has occlusive properties that would undesirably enhance percutaneous transmission of the local anesthetic agent through the skin or mucosa into the systemic circulation, however, the usage and amount of water in the present invention should be minimized. Greater amounts of water will also cause the local anesthetic agent and/or penetration enhancer to precipitate from the composition.

In one embodiment of the present invention, the amount of water in the total composition is from about 0.01 to 20 wt %. In another embodiment, the amount of water is about 0.01 wt % of the total composition.

Alternatively, the topical anesthetic composition contains less than about 10 wt % water. In another embodiment of the present invention, the topical anesthetic composition contains less than about 5 wt % water.

Preparation of the Topical Anesthetic Composition

The topical anesthetic composition of the present invention may be prepared using ordinary production methods. In one embodiment, the local anesthetic agent, penetration enhancer and solvent are introduced into a standard preparation vessel and mixed to form a homogeneous solution. In an alternative embodiment, water is also added to the preparation vessel.

The solution can then be transferred to a suitable packaging container. Suitable packaging containers include a 15 ml polyethylene terephthalate (PET) cartridge with a 20 mm crimp, an 8 ml cartridge with a 13 mm crimp, and 8 ml glass cartridge with an 13 mm crimp, an 8 ml aluminum cartridge with a 13 mm crimp or an 30 ml aluminum tube cartridge with a 20 mm crimp.

A suitable spray device, such as a spray pump with an appropriately sized nozzle, capable of spraying the homogenous solution onto the intended surface can be attached to the packaging container. Spray pumps capable of delivering a constant and steady stream of the topical anesthetic composition without clogging can be used in the present invention. Examples of suitable pump sprayers include those available from Emsar S.p.A. such as the Emsar 32 MSL fragrance and crimp pump sprayer (Emsar, Chieti, Italy) and the Tenex pump sprayer (Bespak, Gary, N.C.). Both pump sprayers have a 70/130 microliter (mcl) capacity and deliver 80 to 120 mg per spray.

Application and Delivery of the Topical Anesthetic Composition

In one embodiment of the present invention, local anesthesia is obtained by topical application of the topical anesthetic composition onthe intended skin surface. Prior to application of the topical anesthetic composition, the intended surface should be cleansed with an appropriate solvent, detergent or by abrasive means. In one embodiment of the present invention, the intended surface is swabbed with an appropriate alcohol solution such as commercially available 2-propanol (also referred to as isopropyl alcohol) or ethanol. The intended surface can also be prepared using soap and water.

The topical anesthetic composition is a homogeneous liquid solution and therefore the topical anesthetic composition can be sprayed onto the intended skin surface using a suitable pump sprayer. The term spraying, as used herein, refers to dispersing the topical anesthetic composition onto an intended surface by propelling the topical anesthetic composition in a stream of droplets. The intended surface can be skin, either intact or damaged, or mucosa. The pump sprayer delivers an effective dosage amount of about 30 mg/in² of the local anesthetic agent where the pump sprayer is held about 1 inch from the intended surface. Alternatively, the pump sprayer delivers an effective dosage amount of about 15 mg/in² where the pump sprayer is held about 2 inches from the intended surface. Efficacy of the topical anesthetic composition using this application method is from about 3 to 15 minutes. In an alternative embodiment, the efficacy of the topical anesthetic composition using this application method is from about 5 to 10 minutes. The duration of the effect is sustained for about 30 minutes. In an alternative embodiment, the duration of the effect is sustained for from about 10 to 12 minutes.

Examples of procedures that can be perfommed on skin or mucosa that can be anesthetized according to the present invention include needle insertion, circumcision, incision, punch biopsy, nevi excision, dental work, toothache and relief of teething pain in infants and the like.

### EXAMPLES

EXAMPLE 1
Topical anesthetic composition

| | |
|---|---|
| Local Anesthetic Agent: | Lidocaine Base available from |
| | Hawkins, Inc., Minneapolis, MN |
| Penetration Enhancer: | Isopropyl Palmitate available |
| | from Cognis, Cincinnati, OH |
| Anhydrous Volatile Solvent: | SDA-40B Ethanol available |
| | from Aaper Alcohol, |
| | Shelbyville, KY |
| | SDA-39B Ethanol available |
| | from Aaper Alcohol |
| | 190 Ethanol available from |
| | Aaper Alcohol |
| | 200 Ethanol available from |
| | Aaper Alcohol |

### Example 1

### Topical Anesthetic Compositions

| | | Composition | | | | | | |
|---|---|---|---|---|---|---|---|---|
| | | (wt % of total) | | | | | | |
| Component | | A | B | C | D | E | F | G |
| Solvent | SDA-40B Ethanol | 50.0 | | | | | 60.0 | |
| (Excipient) | SDA-39C Ethanol | | 50.0 | | | | | 55.0 |
| | 190 Ethanol | | | 50.0 | | 55.0 | | |
| | 200 Ethanol | | | | 50.0 | | | |
| Anesthetic | Lidocaine Base | 35.0 | 35.0 | 35.0 | 35.0 | 35.0 | 35.0 | 35.0 |
| Agent | | | | | | | | |
| Penetration | Isopropyl | 10.0 | 10.0 | 10.0 | 10.0 | 10.0 | 5.0 | 5.0 |
| Ehanhancer | Palmitate | | | | | | | |
| Water | Water | 5.0 | 5.0 | 5.0 | 5.0 | | | 5.0 |
| (Excipient) | | | | | | | | |

| | | | | | | | |
|---|---|---|---|---|---|---|---|
| Total | 100.0 | 100.0 | 100.0 | 100.0 | 100.0 | 100.0 | 100.0 |

### Example 2

### Performance Results

Two sprays of Composition A were applied to intact skin on the back of the left hand to deliver 29.4 mg of lidocaine base.

A skin stick test was conducted five minutes after application of Composition A. Ten sticks were performed on intact skin on the back of the left hand using a syringe with a 28G needle. No pain was felt during the first nine sticks in the treated area where the Composition A had been applied. Only a slight sensation of pressure was observed. The tenth stick was performed outside of the treated area and pain was felt as the needle penetrated the untreated skin.

The skin stick test was repeated 30 minutes after application of Composition A. This time, five sticks were performed on intact skin on the back on the left hand using a syringe with a 28G needle. No pain was felt in the treated area where the Composition A had been applied.

A week later, a second skin stick test was conducted. Two sprays of Composition A were applied to intact skin on the back of the left hand to deliver 29.4 mg of lidocaine base.

A skin stick test was conducted three minutes after application of Composition A. No pain was felt in the treated area.

The skin stick test was repeated for Compositions B, C, and D. Each of the Compositions produced the same results as those observed for Composition A.

The skin stick test was also repeated for Compositions F and G. A skin stick test was conducted ten minutes after application of Composition F and G. No pain was felt in the treated area for either Composition F or G.

Although the present invention has been described with reference to specific embodiments. Workers skilled in the art, however, will recognize that changes may be made in form and detail without departing from the spirit and scope of the invention. In addition, the invention is not to be taken as limited to all of the details thereof as modifications and variations thereof may be made without departing from the spirit or scope of the invention.

1. A topical anesthetic composition for administration to skin or mucosa consisting essentially of a sprayable homogeneous solution having:
(a) one or more local anesthetic agents, wherein the concentration of the local anesthetic agent is from about 5 to about 50 wt % of the topical anesthetic composition;
(b) penetration enhancer, wherein the concentration of the penetration enhancer is from about 1 to about 30 wt % of the topical anesthetic composition; and
(c) an anhydrous volatile solvent, wherein the concentration of the anhydrous volatile solvent is from about 10 to about 94 wt % of the topical anesthetic composition.
(c) water, wherein the concentration of the water is from about 0.01 to about 20 wt % of the topical anesthetic composition.

2. The topical anesthetic composition of claim 1, wherein the concentration of the local anesthetic agent is about 35 wt %.

3. The topical anesthetic composition of claim 1, wherein the local anesthetic agent is selected from the group consisting of lidocaine, tetracaine, benzocaine, procaine, mepivacaine, bupivacaine, etidocaine, cocaine and mixtures thereof.

4. The topical anesthetic composition of claim 1, wherein the local anesthetic agent is lidocaine.

5. The topical anesthetic composition of claim 1, wherein the concentration of the penetration enhancer is about 10 wt %.

6. The topical anesthetic composition of claim 1, wherein the penetration enhancer is a fatty acid ester selected from the group consisting of isopropyl palmitate, isopropyl myristate, isopropyl laurate, diisopropyl adipate, ester derivatives of capric acid, lauric acid, leucinic acid, neodecanoic acid, and oleic acid, sorbitan esters, long chain alkyl esters of 2-pyrrolidone, and combinations thereof.

7. The topical anesthetic composition of claim 6, wherein penetration enhancer is isopropyl palmitate.

8. The topical anesthetic composition of claim 1, wherein the concentration of the anhydrous volatile solvent is from about 50 to about 55 wt % of the topical anesthetic composition.

9. The topical anesthetic composition of claim 1, wherein the anhydrous volatile solvent is ethanol.

10. The topical anesthetic composition of claim 1, wherein the concentration of water is about 0.01 wt %.

11. The topical anesthetic composition of claim 1, wherein the unit dosage of the local anesthetic agent is about 30 mg/in² when sprayed 1-inch from an intended surface.

12. The topical anesthetic composition of claim 1, wherein the topical anesthetic composition has an efficacy of from about 3 to 15 minutes.

13. The topical anesthetic composition of claim 1, wherein the topical anesthetic composition has an efficacy of from about 5 to 10 minutes.

14. The topical anesthetic composition of claims 1, wherein the topical anesthetic composition remains effective for about 30 minutes.

15. A topical anesthetic composition for administration to skin or mucosa consisting essentially of a sprayable homogeneous solution having:
(a) one or more local anesthetic agents, wherein the concentration of the local anesthetic agent is from about 5 to about 50 wt % of the topical anesthetic composition;
(b) penetration enhancer, wherein the concentration of the penetration enhancer is from about 1 to about 30 wt % of the topical anesthetic composition; and
(c) an anhydrous volatile solvent, wherein the concentration of the anhydrous volatile solvent is from about 10 to about 94 wt % of the topical anesthetic composition.
(c) water, wherein the concentration of the water is less than about 10 wt % of the topical anesthetic composition.

16. The topical anesthetic composition of claim 16, wherein the concentration of the water is less than about 5 wt %.

17. The topical anesthetic composition of claim 16, wherein the concentration of the water is less than about 1 wt %.

18. A topical anesthetic composition for administration to skin or mucosa consisting essentially of a sprayable homogeneous solution having:
(a) lidocaine base, wherein the concentration of lidocaine base is from about 5 to about 50 wt % of the topical anesthetic composition;
(b) isopropyl palmitate, wherein the concentration isopropyl palmitate is from about 1 to about 30 wt % of the topical anesthetic composition;
(c) ethanol, wherein the concentration of the ethanol is from about 10 to about 94 wt % of the topical anesthetic composition; and
(d) water, wherein the concentration of the water is less than about 10 wt % of the topical anesthetic composition.

19. A method of anesthetizing skin or mucosa, said method comprising:
swabbing the skin or mucosa with alcohol;
spraying a topical anesthetic composition on skin or mucosa consisting essentially of:
(a) lidocaine base, wherein the concentration of lidocaine base is from about 5 to about 50 wt % of the topical anesthetic composition;
(b) isopropyl palmitate, wherein the concentration isopropyl palmitate is from about 1 to about 30 wt % of the topical anesthetic composition;
(c) ethanol, wherein the concentration of the ethanol is from about 10 to about 94 wt % of the topical anesthetic composition; and
(d) water, wherein the concentration of the water is less than about 10 wt % of the topical anesthetic composition.

20. The method of claim 20, wherein a spray pump is used to spray the topical anesthetic composition onto skin or mucosa.

21. The method of claim 20, wherein about 30 mg/in² of the topical anesthetic composition is administered to the skin or mucosa where the spray pump is held 1-inch from the intended surface.

22. The method of claim 20, wherein the skin or mucosa is anesthetized within from about 3 to about 15 minutes after administration of the topical anesthetic composition.

23. The method of claim 20, wherein the skin or mucosa is anesthetized within from about to about 10 minutes after administration of the topical anesthetic composition.

24. The method of claim 20, wherein the skin or mucosa remains anesthetized for about 30 minutes.
